(19) United States
(12) Patent Application Publication
(10) Pub. No.: US 2021/0369777 A1
(43) Pub. Date: Dec. 2, 2021

(54) THERAPEUTIC COMPOSITIONS AND RELATED METHODS

(71) Applicant: BRAHM HOLDINGS, LLC, Germantown, TN (US)

(72) Inventor: Timothy R. BRAHM, Germantown, TN (US)

(21) Appl. No.: 17/331,832

(22) Filed: May 27, 2021

Related U.S. Application Data
(60) Provisional application No. 63/143,213, filed on Jan. 29, 2021.
(60) Provisional application No. 62/704,747, filed on May 27, 2020.

(51) Int. Cl. A61K 35/17; A61K 38/19; A61K 38/21; A61K 45/06

(52) U.S. Cl. CPC A61K 35/17; A61K 45/06; A61K 38/217; A61K 38/191

(57) ABSTRACT

A therapeutic composition is provided. The therapeutic composition is particularly useful in the treatment of various symptoms arising from immune responses to pathogenic infections. The therapeutic compositions includes an acellular mammalian birth tissue material composition and a stem cell composition. Methods of treatment are also provided.

### CROSS-REFERENCE TO RELATED APPLICATIONS

The present application claims priority to U.S. Application Ser. No. 62/704,747 filed May 27, 2020 and U.S. Application Ser. No. 62/143,214 filed Jan. 29, 2021

### TECHNICAL FIELD

The present disclosure relates to therapeutic compositions useful to treat symptoms associated with an immune response as well as treat pathogenic infections.

### BACKGROUND OF THE DISCLOSURE

Pathogenic infections continue to cause pandemics resulting the loss of life and severe economic impact. Pharmaceuticals that are often used to help relieve aches, pains, or other symptoms. While these pharmacotherapies may help to reduce symptoms, such therapies are not a permanent solution and may simply mask underlying, lasting damage.

Pain and inflammation are but two exemplary symptoms that can be triggered by activation of nociceptive and thermal-sensitive nerve endings in tissues and may stem from a pathogenic infection. Such activation can be caused by tissue injury, viral infection, or innate conditions, such as autoimmune disease. For example, once an individual has been infected with the herpes virus, the virus will thereafter remain latent in the body. In the latent state, the virus can settle in nerve cell bodies in the ganglia. Stimuli, such as influenza infection, other respiratory disorders, gastrointestinal infections, stress, fatigue, menstruation, pregnancy, allergy, sunlight, or fever, can activate the latent virus, which may then travel from the ganglia to the skin surface and multiply, causing various symptoms. Other symptoms of pathogenic infections include pain, neurogenic inflammation, blistering, scarring of internal organs (e.g., lungs), cytokine storm, and other somatosensory system manifestations such as, for example, pain, itch, tickle, tingle, and numbness.

At the onset of such symptoms, conventional methods for the treatment of pain and inflammation are often initiated, for example, non-steroidal anti-inflammatory drugs (NSAIDs), antidepressants, and antiviral medications (e.g., acyclovir, famciclovir, or valacyclovir). Such conventional methods often fall short of true treatment by only providing temporary relief, masking symptoms and/or causing serious side effects from prolonged use.

### SUMMARY OF DISCLOSURE

A therapeutic composition is provided. The therapeutic composition is particularly useful in the treatment of various symptoms arising from immune responses to pathogenic infections. The therapeutic compositions includes an acellular mammalian birth tissue material composition; and a stem cell composition. According to one embodiment, the therapeutic composition further includes one or more tissue-remodeling biomolecules, one or more proliferation biomolecules, one or more angiogenic biomolecules, one or more migration biomolecules, one or more anti-inflammatory biomolecules, one or more anti-microbial biomolecules, or a combination thereof. According to one embodiment, the stem cell composition includes one or more natural killer cells. According to one embodiment, the natural killer cells are derived from allogenic or autologous sources. According to one embodiment, the natural killer cells are derived from mammalian blood, NK-92 cells, pluripotent cells, human induced pluripotent stem cell-derived natural killer (hnCD16-iNK) cells, peripheral blood, human embryonic stem cells (hESCs), bone marrow, any one or more immune cells, one or more engineered cells, chimeric antigen receptor T (CAR T) cells, umbilical cord blood, or a combination thereof.

According to one embodiment, the source of the stem cell composition is autologous or derived from the same mammal receiving treatment with the same. According to one embodiment, the source of the natural killer cells is autologous or derived from the same mammal receiving treatment with the same. According to one embodiment, the mammalian blood is autologous or derived from the same mammal receiving treatment with the same. According to one embodiment, the source of the mammalian birth tissue is autologous or derived from the same mammal receiving treatment with the same. By receiving autologous sourced therapeutic composition components, the need to combine different sources is eliminated.

According to one embodiment, the source of the stem cell composition is derived from a mammal different from the mammal receiving treatment (i.e., allogenic). According to one embodiment, the mammalian blood is derived from a mammal different from the mammal receiving treatment (i.e., allogenic). According to one embodiment, the source of the mammalian birth tissue is derived from a mammal different from the mammal receiving treatment (i.e., allogenic). By receiving therapeutic composition components from a different source, an immune cell response is elicited.

According to one embodiment, the donor of the mammalian birth tissue or stem cell compositions components source is seropositive (i.e., shows presence of antibodies) for targeted pathogens. By being seropositive, the resulting therapeutic composition sensitized to treat pathogens in recipients in need of treatment.

According to another aspect, a method of preparing a therapeutic composition is provided. The method includes the steps of:

obtaining mammalian birth tissue material;

decellularizing the mammalian birth tissue material to form an acellular mammalian birth tissue material composition;

forming a stem cell composition by isolating supernatant obtained from centrifugation of a source of one more stem cells;

combining the stem cell composition with the mammalian birth tissue material composition to form a therapeutic composition. The stem cell composition may include one or more natural killer stem cells or natural killer stem cell supernatant as provided herein.

According to another aspect, a method of treating inflammation, pain, scarring, or cytokine storm arising from a pathogen infection on or within a mammalian body is provided. The method includes the step of administering a therapeutically effective amount of the therapeutic composition as provided herein to a subject in need of treatment. According to one embodiment, the scarring is located in or on one or more internal organs. According to one embodiment, the one or more internal organs includes lungs.

According to another aspect, a method of treating a pathogenic infection on or within a mammalian body is provided. The method includes the step of administering a therapeutically effective amount of the therapeutic composition as provided herein to a subject in need of treatment. Upon administration, the pathogen is inactivated thereby eliminating the need for an immune response. According to one embodiment, the pathogen is a virus. According to one embodiment, the virus is a coronavirus or influenza.

### DETAILED DESCRIPTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

As used herein, “mammalian birth tissue material” encompasses one or more of the components of the placental organ including, but not limited to, the placental globe, the umbilical cord, the umbilical cord blood, the chorionic membrane, the amniotic membrane, the Wharton's jelly, other gelatins, cells, and extracellular material, and the amniotic fluid.

As used herein, “placental tissue components” encompasses one or more of the tissue components of the placental organ including, but not limited to, the placental globe, the umbilical cord, the umbilical cord blood, the chorionic membrane, the amniotic membrane, the Wharton's jelly and other gelatins, cells and extracellular material.

As used herein, the term “amnion” and “amniotic membrane” are used interchangeably.

As used herein, the term “effective amount” or “therapeutically effective amount” refer to an amount of the mammalian birth tissue material composition sufficient to elicit the desired pharmacological or therapeutic effects, thus resulting in an effective treatment of a disease, condition, disorder, or the inflammatory or pain symptoms associated therewith. The effective amount can vary, depending upon factors such as the condition of the patient, the severity of the symptoms of the disorder, and the manner in which the therapeutic composition is administered.

As used herein, the term “treat,” “treatment” or “treating” refers to: (a) the inhibition, reduction, delay or prevention of the onset or progression of the disease, condition, or disorder itself; (b) the delay or prevention of the onset or progression of symptoms (e.g., pain, inflammation) associated with the disease, condition, or disorder; and (c) the reversal of damage caused by an immune response. Treatment may also be manifested by a decrease or elimination of symptoms, reversal of the progression of the disorder, as well as any other contribution to the well-being of the patient.

As used herein, the term “treatment of pain” and the like refers to complete elimination of pain; noticeable reduction of pain by the subject suffering pain; detectable reduction of pain or indicia of pain by objective criteria (e.g., heart rate, blood pressure, muscle tone); or a combination thereof.

As used herein, the term “mucosal membrane” refers to the mucosa of the nose, mouth, eye, ear, vagina or rectum.

As used herein, the term “pathogen,” “pathological,” “pathological contaminant” and “pathological organism” refer to any bacterium, virus or other microorganism (fungi, protozoa, etc.) that can cause disease for a member of the plant or animal kingdom.

As used herein, “tissue-remodeling biomolecules” means biomolecules that are implicated in the reorganization or renovation of existing tissues. The one or more tissue-remodeling biomolecules may comprise: cystatin B (CSTB); cystatin C (CST3); plasminogen activator inhibitor-1 (PAI-1); matrix metallopeptidase 1 (MMP1); matrix metallopeptidase 13 (MMP13); nidogen-1 (NID1); cathepsin L (CTSL); clusterin (CLU); extracellular matrix metalloproteinase inducer (EMMPRIN); TIMP metallopeptidase inhibitor 1 (TIMP1); TIMP metallopeptidase inhibitor 2 (TIMP2); decorin (DCN); or a combination thereof.

As used herein, “proliferation biomolecules” means biomolecules that are implicated in the growth of new tissue. The one or more proliferation biomolecules may comprise: erb-b2 receptor tyrosine kinase 2 (ERBB2); dipeptidyl peptidase 4 (DPP4); epidermal growth factor receptor (EGFR); macrophage-colony stimulating factor (MCSF); activated leukocyte cell adhesion molecule (ALCAM); or a combination thereof.

As used herein, “angiogenic biomolecules” means biomolecules that are implicated in the formation of new blood vessels. The one or more angiogenic biomolecules may comprise: pentraxin 3 (PTX3); angiogenin (ANG); fms related tyrosine kinase 1 (FLT1); thrombospondin 1 (THBS1); urokinase-type plasminogen activator (uPA); transforming growth factor beta induced (TGFBI); or a combination thereof.

As used herein, “migration biomolecules” means biomolecules that are implicated in the movement of cells to specific locations for tissue formation, wound healing and immune responses. The one or more migration biomolecules may comprise: syndecan 4 (SDC4); neuronal cell adhesion molecule (NRCAM); dickkopf WNT signaling pathway inhibitor 3 (DKK3); angiotensinogen (AGT); or a combination thereof.

As used herein, “anti-inflammatory biomolecules” means biomolecules that are implicated in the reduction of inflammation. The one or more anti-inflammatory biomolecules may comprise: follistatin like 1 (FSTL1); galectin 1 (LGALS1); or a combination thereof.

As used herein, “anti-microbial biomolecules” means biomolecules that are implicated in the killing of microorganisms or inhibition of their growth. The one or more anti-microbial biomolecules may comprise: beta-2-microglobulin (B2M).

As used herein, “osteogenesis biomolecules” means biomolecules that are implicated in the formation of bone. The composition may further comprise one or more osteogenesis biomolecules. The one or more osteogenesis biomolecules may comprise: follistatin like 3 (FSTL3); growth differentiation factor 15 (GDF15); or a combination thereof.

As used herein, “pro-inflammatory biomolecules” means biomolecules that are implicated in the promotion of inflammation and related inducement of an immune response. The composition may further comprise one or more pro-inflammatory biomolecules. The one or more pro-inflammatory biomolecules may comprise: tumor necrosis factor receptor 1 (TNFR1).

As used herein, “pro-apoptotic biomolecules” means biomolecules that are implicated in promoting or causing apoptosis in cells. The composition may further comprise one or more pro-apoptotic biomolecules. The one or more pro-apoptotic biomolecules may comprise: Fas cell surface death receptor (FAS).

### Therapeutic Compositions

According to one embodiment, the therapeutic compositions provided include a mammalian birth tissue material composition. According to a particular embodiment, the mammalian birth tissue composition may include one or more placental tissue components including, but not limited to, amnion, chorion, amniotic membrane, amniotic fluid, and Wharton's Jelly. According to one embodiment, the therapeutic composition includes an acellular mammalian birth tissue composition. According to one embodiment, the mammalian source is a human.

According to one embodiment, the therapeutic composition includes one or more tissue-remodeling biomolecules; one or more proliferation biomolecules; one or more angiogenic biomolecules; one or more migration biomolecules; one or more anti-inflammatory biomolecules; and one or more anti-microbial biomolecules. According to one embodiment, the composition includes one or more irradiated components.

According to one embodiment, the therapeutic composition can optionally include one or more bioactive agents such as physiologically compatible minerals, growth factors, wound healing agents (e.g., cytokines including but not limited to PDGF, TGF, and thymosin), hyaluronic acid, wound sealants (such as fibrin with or without thrombin), cellular attractant and scaffolding reagents (e.g., fibronectin) antibiotics, chemotherapeutic agents, antigens, antibodies, enzymes, vectors for gene delivery and hormones.

According to one embodiment, the therapeutic composition can optionally include a suitable carrier to form a composition suitable for treatment or therapy in a mammalian body. According to one embodiment, the carrier composition includes one or more vitamins, minerals, proteins, fats, collagens (including collagen extracted from the placental globe), hyaluronic acid, waxes, glycols and derivatives thereof, glyercols and derivatives thereof, oils (including essential oils), fatty acids, cholesterols, alcohols, emollients, adsorbents, lubricants, emulsifying agents, thickening agents, humectants, surfactants, pharmaceutical ingredients, preservatives, antifungal agents, antioxidants, antimicrobial agents, structuring agents, dispersing agents, pH-adjusting components, sequestering or chelating agents, wetting agents, coloring agents, and other components known in the art to be suitable for use in a composition that can be applied onto or within the human body. The optional carrier composition can be formulated in such a way that the combination of the mammalian birth tissue material composition and the carrier composition are chemically compatible and do not form complexes which precipitate from the final composition.

According to one embodiment, the therapeutic composition includes a stem cell composition. According to one embodiment, the stem cell composition includes one or more natural killer cells or related components. According to one embodiment, the therapeutic composition includes one or more allogenic natural killer cells or related components. According to one embodiment, the therapeutic composition includes one or more autologous natural killer cells or related components.

According to a particular embodiment, the natural killer cells or related components as provided herein are derived from mammalian blood, NK-92 cells, pluripotent cells, human induced pluripotent stem cell-derived natural killer (hnCD16-iNK) cells, peripheral blood, human embryonic stem cells (hESCs), bone marrow, any one or more immune cells, one or more engineered cells, chimeric antigen receptor T (CAR T) cells, or umbilical cord blood. According to a particular embodiment, the one or more natural killer cells or related components is derived from stem cells obtained from the peripheral blood of a young male human donor.

According to one embodiment, the therapeutic composition includes one or more stem cells or other similar component that increases the potency of the therapeutic composition. According to one embodiment, the therapeutic composition includes one or cytokines associated with immune regulation (ENA-78 [CXCL 5] and ICAM-2).

According to one embodiment, the source of the stem cell composition is autologous or derived from the same mammal receiving treatment with the same. According to one embodiment, the source of the natural killer cells is autologous or derived from the same mammal receiving treatment with the same. According to one embodiment, the mammalian blood is autologous or derived from the same mammal receiving treatment with the same. According to one embodiment, the source of the mammalian birth tissue is autologous or derived from the same mammal receiving treatment with the same. By receiving autologous sourced therapeutic composition components, the need to combine different sources is eliminated.

According to one embodiment, the source of the stem cell composition is derived from a mammal different from the mammal receiving treatment (i.e., allogenic). According to one embodiment, the mammalian blood is derived from a mammal different from the mammal receiving treatment (i.e., allogenic). According to one embodiment, the source of the mammalian birth tissue is derived from a mammal different from the mammal receiving treatment (i.e., allogenic). By receiving therapeutic composition components from a different source, an immune cell response is elicited.

According to one embodiment, the donor of the mammalian birth tissue or stem cell compositions components source is seropositive (i.e., shows presence of antibodies) for targeted pathogens. By being seropositive, the resulting therapeutic composition sensitized to treat pathogens in recipients in need of treatment.

According to one embodiment, the therapeutic composition includes one or more osteoclasts. Such osteoclasts are a type of bone cell, derived from hematopoietic stem cells. Their function, resorbing bone tissue, is critical for the maintenance, repair, and remodeling of bones. Bone homeostasis is achieved when there is a balance between osteoblast bone formation and osteoclast bone resorption. Osteoclasts mature through stimulation from osteoblasts expressing RANKL, and their interaction, mediated by firm adhesion via ICAM-1. Osteoclasts also express many ligands for receptors present on activated NK cells. They reported that osteoclasts express ULBP-1, ULBP-2/5/6 and ULBP-3, but little or no MIC-A, MIC-B, or MHC class I-like ligands for NKG2D, the activating receptor of NK cells. Osteoclasts may activate natural killer cell expansion and function (Tseng et al. (2015) Oncotarget 6(24):20002-25). Additionally, osteoclasts secrete significant amounts of IL-12, IL-15, IFN-γ and IL-18, which are known to activate natural killer cells. The osteoclasts may be utilized as natural killer activating ligands.

### Methods of Preparation

A method for preparing a therapeutic composition is provided. The method includes the step of obtaining birth tissue material and preparing a birth tissue material composition. The birth tissue material composition as provided herein may be prepared by recovering placental tissue components and amniotic fluid from a seronegative, healthy mammal such as a human. Potential mammalian birth tissue donors are pre-screened during an examination of pre-natal medical records and blood test results. A comprehensive medical history and behavior risk assessment is obtained from the donor prior to donation incorporating U.S. Public Health Service guidelines. Discussions with the physician(s) and/or the donor mother are conducted to identify circumstances that may lead to the exclusion of the donor or donated tissue. Additionally, a physical exam is performed on the donor to determine whether there is evidence of high risk behavior or infection and to determine the overall general health of the donor.

Infectious disease testing of donor blood specimens is performed for each tissue donor on a specimen collected at the time of donation or within seven days prior to or after donation. Advantageously, the methods that are used to screen for a communicable disease follow the regulations as set forth by the Federal Drug Administration and the American Association of Tissue Banks. Exemplary infectious disease testing includes, but is not limited to, antibodies to the human immunodeficiency virus, type 1 and type 2 (anti-HIV-1 and anti-HIV-2); nucleic acid test (NAT) for HIV-1; hepatitis B surface antigen (HBsAg); total antibodies to hepatitis B core antigen (anti-HBc—total, meaning IgG and IgM); antibodies to the hepatitis C virus (anti-HCV); NAT for HCV; antibodies to human T-lymphotropic virus type I and type II (anti-HTLV-I and anti-HTLV-II); and syphilis (a non-treponemal or treponemal-specific assay may be performed).

Mammalian birth tissue is preferably recovered from a full-term Cesarean delivery of a newborn. Alternatively, mammalian birth tissue is recovered from a full-term vaginal delivery of a newborn. The subsequent steps of preparing the mammalian birth tissue material are performed in a controlled environment (i.e., certified biological safety cabinet, hood or clean room). Instruments, solutions, and supplies coming into contact with the mammalian birth tissue material during processing are sterile. All surfaces coming in contact with the mammalian birth tissue material intended for transplant are either sterile or draped using aseptic technique.

Once recovered, one or more of the placental tissue components can be removed via a sterile saline solution rinse, blunt dissection, scalpel, or a combination thereof, if necessary. According to one embodiment, the placental globe, umbilical cord, chorionic membrane, and other gelatins, fluids, cells and extracellular matrix are removed and discarded, leaving the amniotic membrane for further processing. In a preferred embodiment, the mammalian birth tissue material is subject to the method of preparation described herein no more than four hours after recovery to preserve cell viability.

The retained placental tissue components can be placed in a sterile transport solution after aseptic recovery. The sterile transport solution is used to provide an advantageous medium to the natural function of the placental tissue components prior to processing. For example, calcium-rich water can be used as the sterile transport solution to provide a medium to drive undifferentiated cells to become osteogenic when implanted. Throughout the preparation of the mammalian birth tissue material, various methods can be used to drive undifferentiated cells to differentiate into specialized cell types including, but not limited to, transport solutions, soaks, particular temperature ranges, and hyperbaric pressure.

The sterile transport solution preferably includes sodium chloride (NaCl) in a concentration range from typically about 10% to typically about 20% by weight. The sterile transport solution can also include one or more of Minimum Essential Medium, Dulbecco's Modified Eagle's Medium, Plasma Lyte-A, human albumin 25% solution, calcium-rich water, alkaline ionized water, or acidic ionized water.

The placental tissue components can be removed from the transport solution and cryopreserved according to methods commonly used in the art. The placental tissue components can be soaked in cryoprotectant prior to cryopreservation. In another embodiment, the cryoprotectant is a commercially available cryoprotectant such as Synth-a-Freeze® available from Invitrogen. Any cryoprotectant specific to the birth tissue material described herein may be used. In one embodiment, cryopreservation is achieved using a controlled rate freezer, resulting in a 1° C. rate from nucleation to −35° C. and a 10° C. per minute cooling rate to a −90° C. end temperature. Any cryopreservation method commonly known in the art may be used.

After cryopreservation, the placental tissue components are subjected to morselization. As used herein, “morselization” means to grind up to particle form. Tissue morselization may occur by any art-recognized method of tissue disruption, including, but not limited to: milling, blending, sonicating, homogenizing, micronizing, pulverizing, macerating, or a combination thereof. In one embodiment, the placental tissue components are subjected to cryogenic milling by methods commonly known in the art. In a preferred embodiment, the tissue is cryogenically milled in a CryoMill° (available from Retsch) for two cycles at a frequency 1/s of 25 Hz with a pre-cooling time of no more than about five minutes, a grinding time of no more than about two minutes, and an intermediate cooling time of no more than about five minutes. In another embodiment, a Freezer/Mill® available from SPEX SamplePrep, LLC may be used. After morselization, the milled placental tissue components can be combined with amniotic fluid or a derivative thereof or cells removed as provided in U.S. Pat. No. 10,039,792 to Brahm, the contents of which are incorporated by reference in its entirety.

According to one embodiment, the method of preparing a therapeutic composition described herein may include the step of decellularizing any mammalian birth tissue material. Such a step may be carried by treating the material with xeno-free enzymes or a porcine trypsin. According to one embodiment, the mammalian birth tissue material may be digested by treatment with one or more animal origin-free, recombinant enzymes used for dissociating a wide range of adherent mammalian cells, including CHO, HEK 293, A529, primary human keratinocytes, and embryonic stem cells. According to a particular embodiment, the human tissue material may be digested in TrypLE digest and incubated about 10 minutes at about 37° C., agitating the flask every 5 minutes. Following first digestion, the mammalian birth tissue material may be moved to the second digestion flask, and incubated for about 30 minutes at about 37° C., agitating every 5 minutes. The first digest solution may then be disposed. After the second TrypLE digest, the birth tissue material may be moved to a third digestion flask and incubated for about 30 minutes at about 37° C., agitating every 5 minutes. The second TrypLE digest solution may be disposed. After the third TrypLE digest is complete, about 100 mL of 1× HBSS may be dded to the digest to dilute the TrypLE, and the container may be swirled to mix the solution. The birth tissue material may then be transferred to a third wash flask of 1× HBSS and the membrane swirled to dilute the TrypLE.

The solution from the third TrypLE digest may be transferred into centrifuge tubes and centrifuged for about 5 minutes at 200×g. The supernatant from each tube may then be aspirated, leaving about 0.5 mL supernatant above any pellet produced. The pellet may be broken apart and triturated to re-suspend the pellet in the remaining supernatant. Any re-suspended cell pellets may optionally be titrated into a single 50 mL tube with about 10 mL of cell culture media to form a cell suspension. The cell suspension may then be filtered through a 70-100 μm cell strainer into a fresh, sterile 50 ml tube.

According to one embodiment, the method of preparing a therapeutic composition includes the step of preparing a stem cell composition. The stem cell composition may be formed by initially collecting or otherwise isolating one or more stem cells or related components cells from the sources described herein. According to a particular embodiment, the one or more stem cells or related components may be obtained from a stem cell supernatant obtained by digesting the stem cell source and centrifugation of the natural killer cell source. Any supernatant formed may then be aspirated, leaving about 0.5 mL supernatant above any pellet produced. The pellet may be broken apart and triturated to re-suspend the pellet in the remaining supernatant. Any re-suspended cell pellets may optionally be titrated into a cell culture media to form a cell suspension. The cell suspension may then be filtered through to form a stem cell composition. According to one embodiment, the stem cell composition may be formed from a source that provides natural killer stem cells as described herein.

### Methods of Treatment

Methods of treatment are also provided. Each of the methods provided herein include the step of administering a therapeutically effective amount of a therapeutic composition as provided herein to a subject in need of treatment. The therapeutic compositions upregulate or otherwise increase the potency of natural killer cells present in the composition. While not being bound by a particular theory, the presence of mammalian birth tissue acts as an adjuvant to upregulate or otherwise increase the potency of natural killer cells present in the composition. The therapeutic compositions upregulate or otherwise double the potency of natural killer cells present in the composition. The therapeutic compositions upregulate or otherwise triple the potency of natural killer cells present in the composition. The therapeutic compositions upregulate or otherwise quadruple the potency of natural killer cells present in the composition.

According to one embodiment, the therapeutic compositions described herein may be administered as a means to deliver any variety of components as provided herein across the blood brain barrier. According to one embodiment, the therapeutic compositions described herein may be administered as a means to deliver one or more cytokines across the blood brain barrier. According to one embodiment, the one or more cytokines includes any variety of cytokines that are beneficial for treatment of a variety of conditions. According to a particular embodiment, the one or more cytokines includes interleukins, lymphokines, monokines, interferons (IFN), colony stimulating factors (CSF), chemokines and a variety of other proteins.

According to a particular embodiment, the therapeutic compositions described herein may be administered as a means to deliver one or more cytokines across the blood brain barrier to treat cancer. According to a particular embodiment, the therapeutic compositions described herein may be administered as a means to deliver one or more cytokines across the blood brain barrier thereby increasing tumor immunogenicity, disrupting proliferative mechanisms, and inhibiting tumor angiogenesis. According to a particular embodiment, the one or more cytokines includes interferon-gamma, tumor necrosis factor, or a combination thereof.

According to one embodiment, the therapeutic composition described herein may be administered by a user (i.e., medical professional) either through injection or by direct application to the chosen site. Modes of administration include, but are not limited to: nebulization/aerosol (e.g., lungs and airways/esophagus), intramuscular, subcutaneous, intraperitoneal, percutaneous, soft tissue injection, intravenous, intravascular, intracerebral, transdermal, intraocular, topical or mucosal. A single injection or multiple injections may be administered which can be to a single site or to more than one site in the subject to be treated. Multiple administrations may occur essentially at the same time or separated in time. Other suitable formulations may include, but are not limited to, an aerosol, cream, emulsion, spray, gel, ointment, salve, butter, gel, putty, balm, or pliable stick. In one embodiment, the gel or putty carrier could be achieved through collagen extracted from human birth tissue.

In another aspect, the therapeutic composition described herein can be administered alone, or in combination with one or more additional structural carriers, including, but not limited to, a placental membrane construct (e.g., amniotic membrane wound covering), a soft tissue allograft, a bone allograft (e.g., FDBA), or platelet rich plasma. The therapeutic composition can be administered alone, or in combination with one or more additional bioactive agents such as physiologically compatible minerals, growth factors, antibiotics, chemotherapeutic agents, antigen, antibodies, enzymes, vectors for gene delivery and hormones.

According to one embodiment, the therapeutic compositions as provided herein may be utilized to treat a pathogenic infection. Particularly, the therapeutic compositions as provided herein may be utilized to kill, weaken, inactivate or otherwise eliminate a pathogen as well as the immune response thereto.

According to one embodiment, the therapeutic compositions as provided herein may be utilized to treat inflammation, pain, or both inflammation and pain arising from a pathogen infection on or within a mammalian body. Examples of pathogens include viruses giving rise to inflammation, pain, or both inflammation and pain within a body include, but are not limited to, viruses of the Coronaviruse family (SARS, MERS COVID19), Influenza (A and B), Herpesviridae family (e.g., herpes simplex virus-1, herpes simplex virus-2, varicella zoster (shingles), Epstein-Barr virus, cytomegalovirus, roseolovirus, pityriasis rosea and Kaposi's sarcoma-associated herpesvirus), viruses of the Poxviridae family (e.g., Molluscum contagiosum virus), human lymphotrophic viruses (e.g., HTLV-1 and HTLV-2), human immunodeficiency viruses (e.g., HIV-1 and HIV-2) and any other virus that may cause an immune response that results in adverse symptoms or a virus that may rest latent within the body of a human or animal and later activate in response to external stimuli. According to one embodiment, the therapeutic compositions may be administered to the lungs via an aerosol or nebulization composition that is inhaled.

According to one embodiment, the therapeutic compositions as provided herein may be utilized to treat scarring either internal or external to a mammal. According to one embodiment, the scarring includes scarring of one more internal organs. Such scarring includes, but is not limited to, scarring of the lungs or other pulmonary fibrosis or injury to the lung. According to one embodiment, the scarring is caused by an immune response to one or viruses as provided herein including, but not limited to, those of the Coronaviruse family (SARS, MERS, COVID19) and influenza (A and B).

According to one embodiment, the therapeutic compositions as provided herein may be utilized to treat a cytokine storm. Such a storm may manifest from a cytokine balance issue causing acute respiratory distress syndrome (ARDS). Such a storm may be caused by one or more viruses such as, for example those of the Coronaviruse family (SARS, MERS, COVID19) and influenza (A and B).

According to one embodiment, the therapeutic compositions as provided herein may be utilized to treat an inflammatory disease of the skin, lung, esophagus, nasal passages or any mucosal membrane. Such inflammatory diseases include, but are not limited to, eczema, psoriasis, acne, seborrhea, seborrheic dermatitis, ichthyosis, ulcers, psoriasis, seborrheic dermatitis of the face and trunk, seborrheic blepharitis, contact dermatitis, stasis dermatitis and exfoliative dermatitis. In other embodiments, the inflammatory disease or condition is an ocular condition, including, but not limited to, conjunctivitis, uveitis, anterior uveitis, glaucoma, scleritis and dry eye syndrome. In other embodiments, the inflammatory disease or condition is a rheumatic disease, including, but not limited to, osteoarthritis, rheumatoid arthritis, juvenile rheumatoid arthritis, fibromyalgia, scleroderma, spondyloarthropathies, gout, infectious arthritis, polymyalgia rheumatica, polymyositis, psoriatic arthritis, bursitis, tendinitis, CIAS1-related Autoinflammatory Periodic Syndromes (CAPS), pelvic inflammatory disease, interstitial cystitis or Henoh-Schonlein purpura. In other embodiments, the inflammatory disease or condition is an inflammatory disorder, including, but not limited to, autoimmune diseases (e.g., systemic lupus erythematosus, Sjogren's syndrome, sarcoidosis, Behcet's syndrome, ankylosing spondylitis, haemolytic autoimmune anaemias, multiple sclerosis and amyotrophic lateral sclerosis), amyloidosis, asthma, atherosclerosis, osteoporosis, bronchitis, enuresis, eosinophilic disease, gastrointestinal disorders (e.g., Inflammatory Bowel Disease, peptic ulcers, regional enteritis, diverticulitis, gastrointestinal bleeding, Crohn's disease, gastritis, diarrhea, irritable bowel syndrome and ulcerative colitis), gastroesophageal reflux disease, eosinophilic esophagitis, gastroparesis such as diabetic gastroparesis, food intolerances and food allergies. According to one embodiment, the inflammatory disease may be caused by one or more viruses such as, for example those of the Coronaviruse family (SARS, MERS, COVID19) and influenza (A and B).

According to one embodiment, the therapeutic compositions as provided herein may be utilized to treat inflammation, pain, or both inflammation and pain arising on or within the skin or mucosal membranes. Such inflammation and pain may manifest in the form of blisters or lesions of the dermis or epidermis and mucosal membranes. For example, such manifestations include canker sores, cold sores, fever blisters and genital lesions. In certain embodiments, inflammation and pain may manifest in the form of pressure, itch, tickle, tingle, sensitization, or numbness. Such symptoms can arise when a virus is situated in the neuronal tissue (e.g., nerve cell bodies in the ganglia). Alternatively, such symptoms may be related to diseases or conditions such as atopy, diabetes, multiple sclerosis, and hypertension. According to one embodiment, the pain and inflammation may be caused by one or more viruses such as, for example those of the Coronaviruse family (SARS, MERS, COVID19) and influenza (A and B). Thus, the methods provided herein are particularly suited for treatment of neurogenic inflammation. By treating neurogenic inflammation, the onset of neuropathic pain is ultimately prevented.

According to one embodiment, the therapeutic compositions as provided herein may be utilized to treat inflammation associated with conditions, including, but not limited to, vascular diseases, migraine headaches, tension headaches, periarteritis nodosa, thyroiditis, aplastic anemia, Hodgkin's disease, sclerodoma, rheumatic fever, type I diabetes, myasthenia gravis, nephrotic syndrome, polymyositis, gingivitis, hypersensitivity, conjunctivitis, multiple sclerosis, and ischemia (e.g., myocardial ischemia). The compounds may be useful for treating neuroinflammation associated with brain disorders (e.g., Parkinson's disease and Alzheimer's disease) and chronic inflammation associated with cranial radiation injury. The compounds may be useful for treating acute inflammatory conditions (such as those resulting from infection) and chronic inflammatory conditions (such as those resulting from asthma, arthritis and inflammatory bowel disease). The compounds may also be useful in treating inflammation associated with trauma and non-inflammatory myalgia.

According to one embodiment, the therapeutic compositions as provided herein may be utilized to treat pain wherein said pain is neuropathic pain. In a specific embodiment, the neuropathic pain is caused by a virus, e.g., varicella zoster, herpes (e.g., herpes simplex) or human immunodeficiency virus (HIV). In a specific embodiment, said neuropathic pain is caused by diabetic neuropathy. In another specific embodiment, said neuropathic pain is caused by injury to a nerve in said individual. In another specific embodiment, said neuropathic pain is caused by a drug. In certain specific embodiments, said drug is or comprises a platinum-containing anticancer drug, e.g., oxaliplatin, carboplatin or cisplatin, or another chemotherapeutic drug such as paclitaxel or vincristine. In another embodiment the pain is cause by radiation injury, e.g., radiation injury that is part of cancer treatment. In another specific embodiment, said neuropathic pain is caused by inflammation, e.g., neuroinflammation, neuritis.

According to one embodiment, the therapeutic compositions as provided herein may be utilized to treat pain wherein said pain is inflammatory pain. In another embodiment, said pain is bone pain. In a specific embodiment, said bone pain is associated with or caused by cancer. In another embodiment, said pain is unresponsive to steroid therapy. In another embodiment, said pain is unresponsive to non-steroidal anti-inflammatory therapy. In another embodiment, said pain is unresponsive to opioid therapy. In another embodiment, said pain is unresponsive to opiate therapy.

According to one embodiment, the therapeutic compositions as provided herein may be utilized to treat inflammation and/or pain associated with muscle conditions such as inflammatory myopathies (e.g., myositis, dermatomyositis, inclusion body myositis, polymyositis) and myofascial pain syndrome (e.g., trigger points).

According to one embodiment, the therapeutic compositions as provided herein may be utilized to treat a tumor or otherwise a cancer.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A therapeutic composition comprising
an acellular mammalian birth tissue material composition; and
a stem cell composition.

2. The therapeutic composition of claim 1, further comprising one or more tissue-remodeling biomolecules, one or more proliferation biomolecules, one or more angiogenic biomolecules, one or more migration biomolecules, one or more anti-inflammatory biomolecules, one or more anti-microbial biomolecules, or a combination thereof.

3. The therapeutic composition of claim 1, wherein the stem cell composition includes one or more natural killer cells.

4. The therapeutic composition of claim 3, wherein the natural killer cells are derived from mammalian blood, NK-92 cells, pluripotent cells, human induced pluripotent stem cell-derived natural killer (hnCD16-iNK) cells, peripheral blood, human embryonic stem cells (hESCs), bone marrow, any one or more immune cells, one or more engineered cells, chimeric antigen receptor T (CAR T) cells, umbilical cord blood, or a combination thereof.

5. The therapeutic composition of claim 1, wherein the stem cell composition includes one or more natural killer cells from an autologous or allogenic source.

6. The therapeutic composition of claim 1, wherein the acellular mammalian birth tissue material composition is derived from an autologous or allogenic source.

7. The therapeutic composition of claim 1, wherein the mammalian birth tissue composition, stem cell composition or a combination thereof is derived from a seropositive source for one or more pathogens.

8. A method of preparing a therapeutic composition comprising the steps of:
obtaining mammalian birth tissue material;
decellularizing the mammalian birth tissue material to form an acellular mammalian birth tissue material composition;
forming a stem cell composition by isolating supernatant obtained from centrifugation of a source of one more stem cells;
combining the stem cell composition with the mammalian birth tissue material composition to form a therapeutic composition.

9. A method of treating inflammation, pain, scarring, or cytokine storm arising from a pathogen infection on or within a mammalian body, the method comprising the step of:
administering a therapeutically effective amount of the therapeutic composition of claim 1 to a subject in need of treatment.

10. The method of claim 9, wherein the scarring is located in or on one or more internal organs.

11. The method of claim 10, wherein the one or more internal organs includes lungs.

12. A method of treating a pathogenic infection on or within a mammalian body, the method comprising the step of:
administering a therapeutically effective amount of the therapeutic composition of claim 1 to a subject in need of treatment,
wherein the pathogen is inactivated thereby eliminating the need for an immune response.

13. The method of claim 12, wherein the pathogen is a virus.

14. The method of claim 13, wherein the virus is a coronavirus or influenza.

15. A method of upregulating natural killer cell activity comprising, the method comprising the step of:
administering a therapeutically effective amount of the therapeutic composition of claim 1 to a subject in need of treatment,
wherein the natural killer cell activity is at least doubled.

16. A method of treating cancer located across the blood brain barrier, the method comprising the step of:
administering a therapeutically effective amount of the therapeutic composition of claim 1 to a subject in need of treatment,
wherein one or more cytokines present in the therapeutic composition increase tumor immunogenicity, disrupt proliferative mechanisms, inhibit tumor angiogenesis, or a combination thereof.

17. The method of claim 16, wherein the one or more cytokines includes interferon-gamma, tumor necrosis factor, or a combination thereof.
